(19) United States
(12) Patent Application Publication
(10) Pub. No.: US 2026/0207184 A1
(43) Pub. Date: Jul. 23, 2026

(54) RETRACTOR FOR RADIAL HEAD PROCEDURE

(71) Applicant: Acumed LLC, Hillsboro, OR (US)

(72) Inventors: Michael A. Zwolinski, Willoughby, OH (US); Lawrence Ehmke, Portland, OR (US); Joseph Pia, Strasburg, PA (US)

(21) Appl. No.: 19/444,653

(22) Filed: Jan. 9, 2026

Related U.S. Application Data
(60) Provisional application No. 63/746,728, filed on Jan. 17, 2025.

(51) Int. Cl. A61B 17/02

(52) U.S. Cl. CPC A61B 17/025

(57) ABSTRACT

Provided herein are retractors for use in radial head procedures. The retractor may include an elongated body including a feature configured to engage a capitellum. The retractor may further include a bifurcated end portion configured to engage a radius bone. The bifurcated end portion may include a first protrusion and a second protrusion. A first free end of the first protrusion and a second free end of the second protrusion may be offset from a plane defined by the elongated body.

### INCORPORATION BY REFERENCE TO ANY PRIORITY APPLICATIONS

The application claims benefit under 35 U.S.C. § 119(e) to U.S. Provisional Patent Application Ser. No. 63/746,728, filed Jan. 17, 2025, entitled “RETRACTOR FOR RADIAL HEAD PROCEDURE”, the disclosure of which is hereby incorporated by reference and made part of this specification as if set forth fully herein in its entirety. Any and all applications for which a foreign or domestic priority claim is identified in the Application Data Sheet as filed with the present application are hereby incorporated by reference under 37 CFR 1.57.

### BACKGROUND

The head of a bone generally includes a rounded extension that is used to form a part of a joint. The head is the main articulating surface with the adjacent bone. When the head of a bone becomes fractured or otherwise damaged, it may be difficult to repair. When the head is irreparable, prosthetic replacement of the head of the bone may be necessary to keep the joint intact.

Specifically, prosthetic replacement of the radial head in the elbow joint may be performed. The elbow joint is comprised of the humerus, the radius, and the ulna. The radial head articulates at its end with the capitellum of the humerus, to form the humeroradial joint, and on its side with the radial notch of the ulna, to form the proximal radioulnar joint.

A radial head replacement may include removing the entirety of the radial head and replacing it with a radial head prosthesis. During replacement, a bore may be drilled into the radius bone and the end of the residual radius bone may be smoothed and/or planed. In order to access the radius bone for boring, planning, and/or insertion of a radial head prosthesis, a retractor may be used to lift the radial bone away from the humerus and the ulna and out of an incision site in the arm. However, current retractors used for elevating the radial bone are smooth, narrow and flat and tend to be unstable during use. There exists a need for a radius bone retractor that provides stability for elevation of the radius bone while allowing use of surgical tools on the residual radius bone.

### SUMMARY

The present disclosure provides a new and innovative radius bone retractor that provides stability when positioning the radius during radial head replacement procedures. The retractor according to the present disclosure may include a handle, a portion configured to engage the humerus bone, and a portion configured to cradle the radius bone. A user may apply a force to the handle, causing the humerus bone to be used as a fulcrum to prop the radius bone away from the ulna and humerus and out of an incision site.

One aspect of the present disclosure provides a retractor for a radial head procedure including an elongated body and a bifurcated end portion. The elongated body may include a feature configured to engage a capitellum, such as an opening. The bifurcated end portion may be configured to engage a radius bone. The bifurcated end portion may include a first protrusion and a second protrusion. A first free end of the first protrusion and a second free end of the second protrusion may be offset from a plane defined by the elongated body. The radius bone may be configured to be placed between the first protrusion and the second protrusion.

In some embodiments, the elongated body may include a first portion defining a first plane, a second portion defining a second plane angled relative to the first plane, and a third portion defining a third plane angled relative to the second plane and substantially parallel to the first plane. The elongated body may further include a feature, such as an opening or an indentation, configured to engage a capitellum. The feature may be on the first portion and/or the second portion of the elongate body.

A third aspect of the present disclosure provides a method for retracting a radius using a radial retractor disclosed herein. The method may include inserting the bifurcated end portion of the radial retractor into an incision site having a radius bone and placing the radial bone between the first protrusion and the second protrusion of the bifurcated end portion of the radial retractor. The method may further include applying a force on the radial retractor to elevate the radial bone away from the capitellum. Applying the force may cause the feature to engage with the capitellum.

Additional features and advantages of the disclosed apparatus and methods are described in, and will be apparent from, the following Detailed Description and the Figures.

### BRIEF DESCRIPTION OF THE DRAWINGS

FIGS. 1A, 1B and 1C illustrate perspective views of a retractor for a radial head procedure, according to an example of the present disclosure.

FIG. 2 illustrates a second example retractor for a radial head procedure, according to an example embodiment of the present disclosure.

FIG. 3 illustrates a third example retractor for a radial head procedure, according to an example embodiment of the present disclosure.

FIGS. 4A and 4B illustrate a fourth example retractor for a radial head procedure from a side view (FIG. 4A) and from a top view (FIG. 4B), according to an example embodiment of the present disclosure.

FIG. 5A illustrates the retractor of FIGS. 4A and 4B as used in a radial head procedure.

FIG. 5B illustrates a second view of the retractor of FIGS. 4A and 4B as used in a radial head procedure.

FIGS. 6A and 6B illustrate an example retractor for a radial head procedure from a side view (FIG. 6A) and from a top view (FIG. 6B), according to an example embodiment of the present disclosure.

FIG. 7 illustrates the example retractor of FIGS. 6A and 6B as used in a radial head procedure.

FIGS. 8A and 8B illustrate an example retractor for a radial head procedure from a side view (FIG. 8A) and from a top view (FIG. 8B), according to an example embodiment of the present disclosure.

FIG. 9 illustrates a top view of the retractor of FIGS. 8A and 8B for a radial head procedure with a modified handle, according to an example embodiment of the present disclosure.

FIGS. 10A to 10D illustrate an example retractor for a radial head procedure from a side view (FIG. 10A), from a top view (FIG. 10B), from a zoomed in view (10C), and from a perspective view (10D) according to an example embodiment of the present disclosure.

### DETAILED DESCRIPTION

The present disclosure is directed to a retractor for a radial head procedure that provides stability and improved access. The retractor may include a handle portion, a portion configured to engage the capitellum, and a portion configured to support the radius. A user may exert a force upon the handle portion, which causes the portion supporting the radius to elevate the radius away from the ulna and the capitellum. The portion configured to engage the capitellum may keep the capitellum out of the way and may also use the capitellum as a fulcrum to raise the retractor upward to expose the radius.

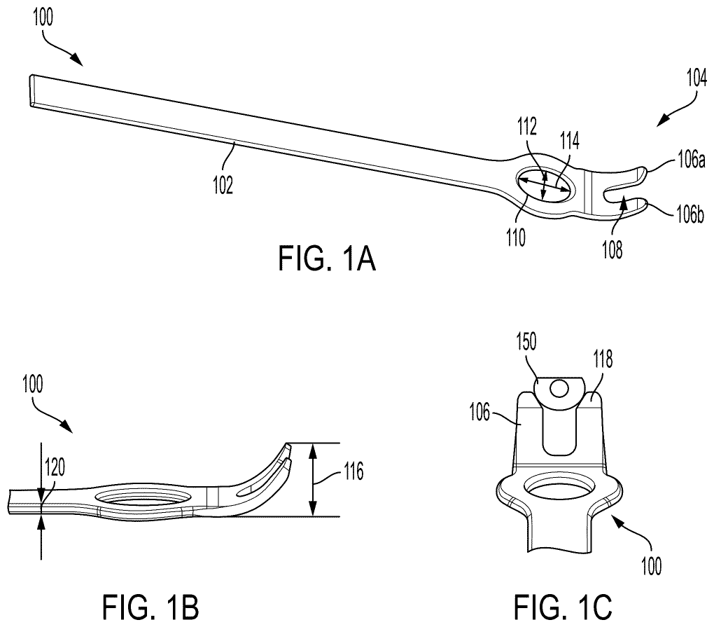

FIGS. 1A to 1C illustrate a retractor 100 according to an example of the present disclosure. As shown in FIG. 1A, the retractor 100 may include an elongated body 102 configured to be grasped by a user. The length of the elongated body 120 may ensure that a user can apply a force outside of the incision site. A portion of the elongated body 102, such as the proximal end portion, may include a feature, such as opening 110, configured to interface with the capitellum or a portion of the humerus. In some embodiments, the feature may include an indentation or bump configured to receive a portion of the humerus.

In some embodiments, the opening 110 is in the shape of an oval with a length 114 greater than a width 112 or a width 112 greater than a length 114. For example, the width 112 of the oval may be between about 0.4 and 0.6 inches and the length 114 may be between about 0.7 and 1.0 inch. However, it can be appreciated that other shapes may be used, such as a circle, a square, a rectangle, or any other desired shape. The opening 110 may be configured to engage the capitellum. For example, the opening 110 may exert a force on the capitellum, which acts as a fulcrum to lift a portion 104 of the retractor 100 configured to support the radius. The opening 110 may also provide stability. For example, a portion of the capitellum may extend through opening 110. An edge of opening 110 may provide friction against the capitellum preventing slippage of the retractor 100 from the bone.

In some embodiments, the portion of the elongated body 102 with the opening 110 is wider than the remainder of the elongated body 102. For example, the portion of the elongate body with opening 110 may have a width between 0.9 and 1.1 inches. The remainder of the elongated body may have a width between 0.25 and 0.5 inches smaller than the portion of the elongated body 102 with the opening 110. In other embodiments, the portion of the elongated body 102 with the opening 110 is the same width or narrower than the remainder of the elongated body 102.

The retractor 100 may include a portion configured to cradle a radius bone, such as bifurcated end portion 104. The portion 104 may extend from an end of the elongated body 102, such as the proximal end portion. The end portion 104 may include a first protrusion 106a and a second protrusion 106b. In some embodiments, first protrusion 106a and second protrusion 106b are substantially parallel to each other. The first protrusion 106a and the second protrusion 106b may be separated by opening 108. The radius bone 150 may be configured to rest between the first protrusion 106a and the second protrusion 106b in opening 108 as depicted in FIG. 1C.

The first protrusion 106a and the second protrusion may include a fixed end (e.g., connected to an end of the elongate body 120) and a free end. As shown in FIG. 1C, a width of the free end portion of the first and the second protrusion 106 a, 106 b may taper 118 toward the free end. This may provide a more stable surface for the radius 150 to rest on during retraction.

The free ends of the first and second protrusions 106a, 106b may be offset from a plane defined by the elongate body 102. In some embodiments, a plane defined by the elongate body 102 extends through a first end portion and a second end portion of the elongate body 102. The first protrusion 106a and the second protrusion 106b may define a plane extending through the free ends and substantially parallel to the plane of the elongate body 102. In some embodiments, a distance 116 between the plane of the elongate body 102 and the plane of the free ends is between about 0.5 inches and 1.0 inch.

As shown in the depicted embodiment, the first protrusion 106a and the second protrusion 106b may curve from the fixed end to the free end. However, in some embodiments, the first protrusion and the second protrusion may include one or more planar surfaces at an angle to each other between the fixed end and the free end.

In some embodiments, a plane extending through the free ends of the first and second protrusion 106a, 106b and a proximal end of the elongate body 102 is transverse to the plane of the elongate body 102. The plane of the protrusions may be at an angle between 115 degrees and 145 degrees, more preferably about 130 degrees, to the plane of the elongate body 102.

The thickness 120 of the retractor 100 may be the same along the entire length (i.e., from the elongate body 102 to the bifurcated end portion 104) of the retractor 100. In some embodiments, the thickness 120 of the retractor, such as the elongated body 102 and/or the first or second protrusion 106a, 106b, may be between about 0.05 and 0.10 inches, more preferably 0.07 inches. In other embodiments, the thickness 120 may vary along the length of the retractor 100. For example, in some embodiments, the thickness of a portion of the first or second protrusion 106a, 106b may be smaller, such as at the free end, than the thickness of the elongated body 102.

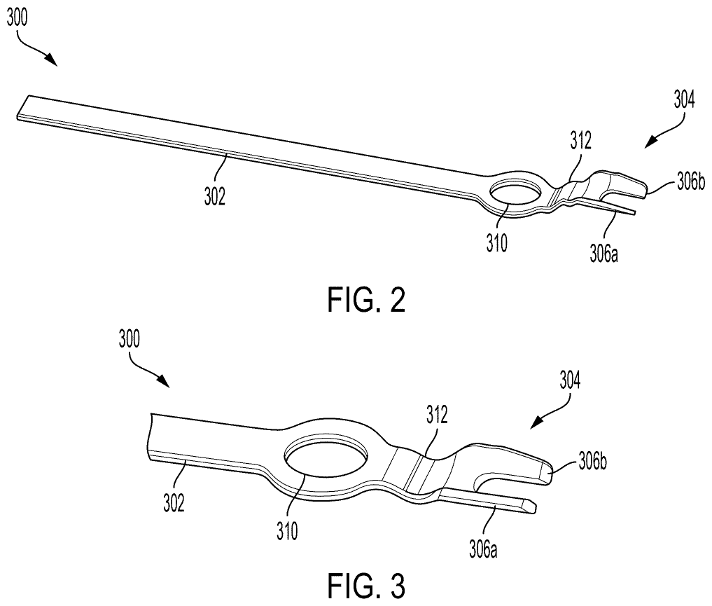

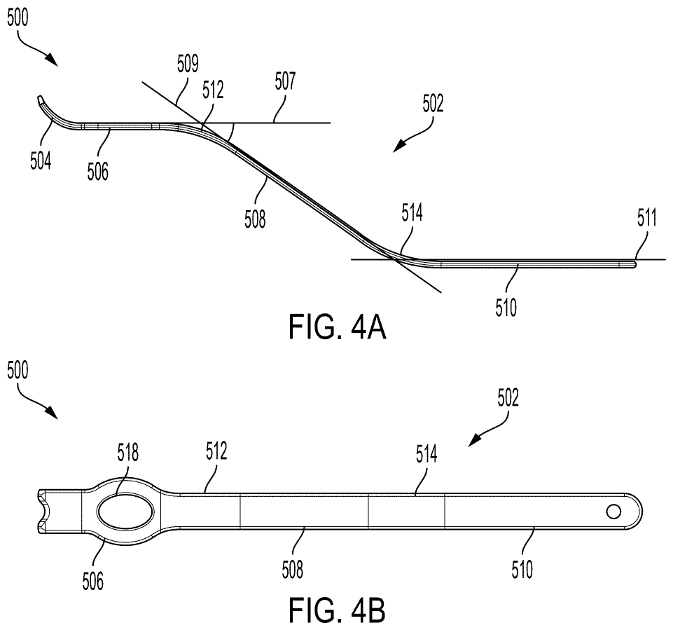

FIGS. 2 and 3 illustrate a retractor 300 according to another example of the present disclosure. The retractor 300 may include an elongate body 302, a feature, such as opening 310, configured to engage the capitellum, and an end portion 304 configured to cradle the radius. The opening 310 and the elongate body 302 of retractor 300 may be the same as the opening 110 and elongate body 102 of retractor 100. In some examples, the end portion 304 of prosthesis 300 may be combined with any of the embodiments of end portion 104 of prosthesis 100.

In some embodiments, retractor 300 include a transition portion 312 between the elongate body 302 and bifurcated end portion 304. The transition portion 312 may provide an offset between the elongate body 302 and the bifurcated end portion 304, such as to prop up the radius bone out of the incision. As shown in FIG. 2, in some embodiments, portion 304 may be offset relative to elongate portion 302 by a high offset, such as between about 5 mm and 25 mm. As shown in FIG. 3, in some embodiments, portion 304 may be offset relative to elongate portion 302 by a low offset, such as between about 1 mm and 10 mm.

As shown in the depicted embodiments, a plane extending through bifurcate end portion 304 may be substantially parallel to a plane extending through the elongated body 302. Bifurcated end portion 304 may include a convex surface from one side of portion 304 to the other side of portion 304 configured to support the radius bone. For example, the radius bone may be configured to lie flat along the bifurcated end portion 304, such as from a fixed end to a free end, as opposed to through an opening between the protrusions. This configuration may provide improved stability of the radius.

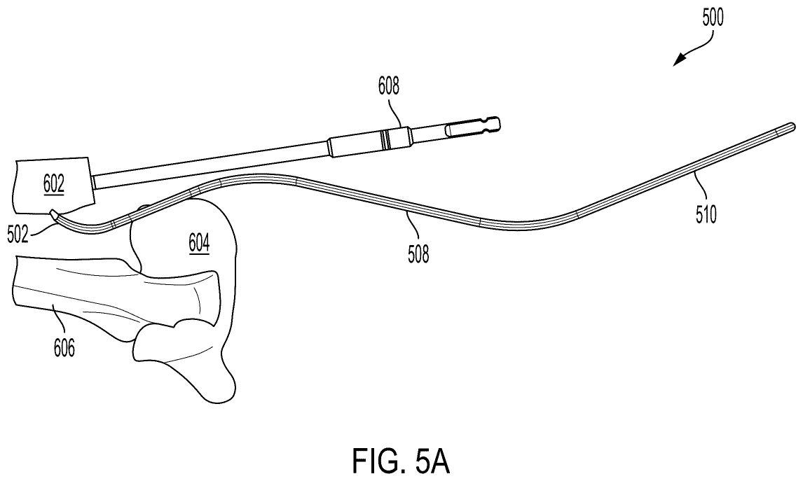

FIGS. 4A and 4B illustrate a retractor 500 according to another example of the present disclosure. The retractor 500 may include an elongate body 502, a feature for engaging the capitellum, such as opening 518, and an end portion 504 configured to cradle the radius. The opening 518 and the end portion 504 of retractor 500 may be the same as the openings 110, 310 and end portions 104, 304 of retractors 100, 300, respectively. In some examples, the elongate body 502 of prosthesis 500 may be combined with any of the embodiments of elongate body 102 of prosthesis 100.

In some embodiments, the elongate body 502 may include one or more bends (e.g., 512, 514). For example, the elongate body may include a first portion 506, a first bend 512, a second portion 508, a second bend 514, and a third portion 510. The first portion 506 may be contiguous with the first bend 512, which is contiguous with the second portion 508, which is contiguous with the second bend 514, which is contiguous with the third portion 510. In some embodiments, the first bend 512 and/or second bend 514 may change the direction of the retractor 500 relative to the portion proximal the respective bend.

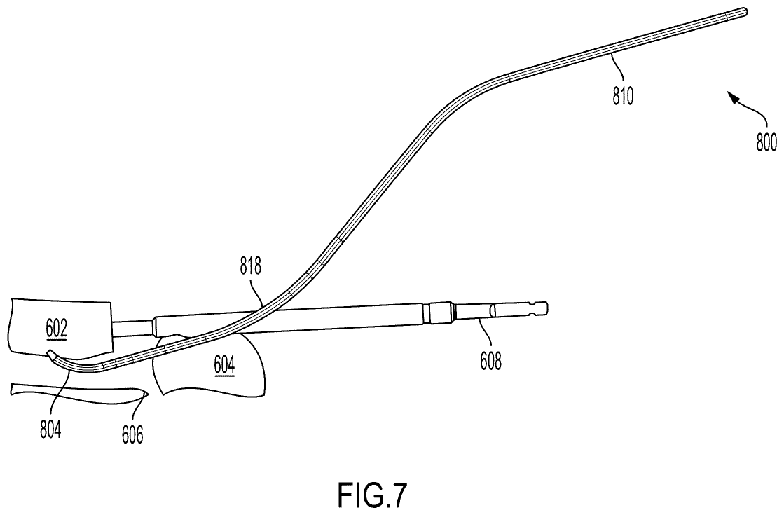

In some embodiments, opening 518 may be in the first portion 506. In other embodiments, opening 518 may extend from the first portion 506 to the second portion 508 as shown in FIG. 6B. In some embodiments, each portion (i.e., 506, 508, 510) may define a plane (i.e., 507, 509, 511) extending either fully or partially through the respective portion. In some embodiments, the plane (i.e., 507, 509, 511) of each or some of the portions (e.g., 506, 508, 510) extends from one end to the other end of each respective portion. As shown in the depicted embodiment, each portion (i.e., 506, 508, 510) or some of the portions may include a generally flat surface. Additionally or alternatively, one or more of the portions (e.g., 506, 508, 510) may include a curved surface (e.g., a concave or convex surface).

In some embodiments, the plane 507 of the first portion 506 may be substantially parallel to the plane 510 of the third portion 510. In some embodiments, the distance between the plane 507 of the first portion 506 and the plane 511 of the third portion 510 is between about 0.5 and 4 inches. In some embodiments, the distance is measured as a vertical distance between one end of the first portion 506 and one end of the third portion 510.

In some embodiments, the plane 509 of the second portion 508 is transverse to the plane 507 of the first portion 506 and/or the third portion 510. The angle between the plane 507 of the first portion 506 and the plane 509 of the second portion 508 may be between about 25 degrees and 45 degrees, more preferably 35 degrees. The second portion 508 may be angled downward from the first portion 506. For example, retractor 500 may include a top surface and a bottom surface, with the bottom surface configured to engage the capitellum. The second portion may extend from the first portion 506 in the direction of the bottom surface as depicted in FIG. 4A. The third portion 510 may extend from the second portion 508 at bend 514.

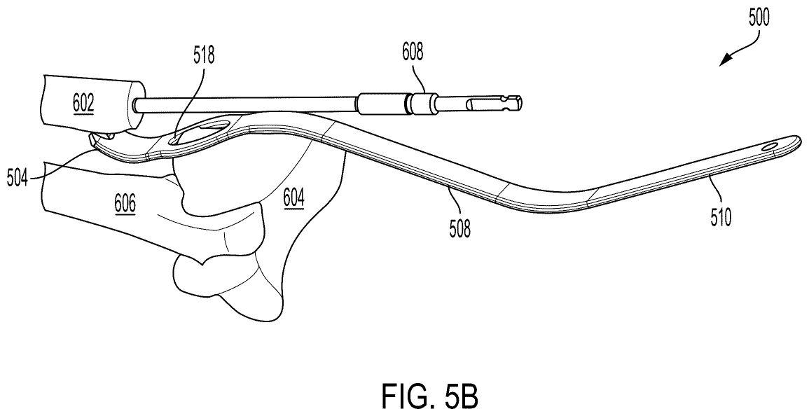

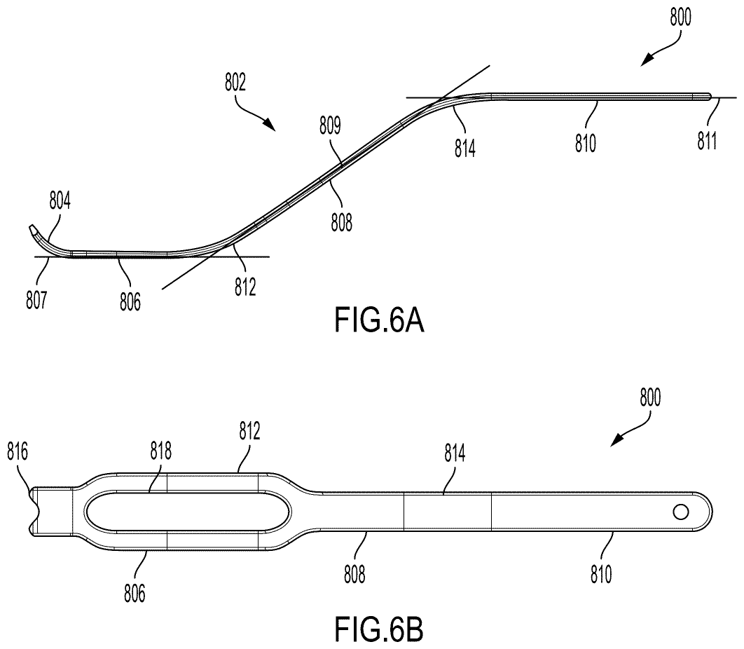

FIGS. 5A and 5B illustrate retractor 500 in use during a radial head procedure. As can be seen from FIG. 5B, opening 518 may be configured to engage the capitellum 604. For example, a portion of the capitellum 604 may traverse through opening 518 of retractor 500. The end portion 504 may be configured to cradle the radius 602, such as a bottom portion of the radius 602. Applying a force to the elongate body 502, such as third portion 510 of elongate body 502, may prop the radius 602 away from the ulna 606 and the capitellum 604 and out of an incision site. In some embodiments, a force is applied on the elongate body 502 toward the arm. The retractor 500 uses the humerus 604 as a leverage point to lift the end portion 504 and the radius 602. A surface of the radius 602 may be exposed for boring, planning, and/or insertion of a radial head prosthesis. For example, as shown in the depicted embodiments, retractor 500 may position the radius 602 to provide easy access for reamer 608 to be inserted into the radius 602 for boring.

FIGS. 6A and 6B illustrate a retractor 800 according to another example of the present disclosure. The retractor 800 may include an elongate body 802, a feature for engaging the capitellum, such as opening 818, and an end portion 804 configured to cradle the radius. The opening 818 and the end portion 804 of retractor 800 may be the same as the openings 110, 310, 518 and end portions 104, 304, 504 of retractors 100, 300, 500 respectively. In some examples, the elongate body 802 of retractor 800 may be combined with any of the embodiments of elongate body 102 of retractor 100.

In some embodiments, the elongate body 802 may include one or more bends (e.g., 812, 814). For example, the elongate body 802 may include a first portion 806, a first bend 812, a second portion 808, a second bend 814, and a third portion 810. The first bend 812 and/or second bend 814 may change the direction of the retractor 800 relative to the portion proximal the respective bend.

In some embodiments, each portion (i.e., 806, 808, 810) may define a plane (i.e., 807, 809, 811) extending either fully or partially through the respective portion. In some embodiments, the plane (i.e., 807, 809, 811) of each or some of portions (e.g., 806, 808, 810) extends from one end to the other end of each respective portion. As shown in the depicted embodiment, each portion (i.e., 806, 808, 810) or some of the portions may include a generally flat surface. Additionally or alternatively, one or more of the portions (e.g., 806, 808, 810) may include a curved surface (e.g., a concave or convex surface).

In some embodiments, the plane 807 of the first portion 806 may be substantially parallel to the plane 809 of the third portion 810. In some embodiments, the distance between the plane 807 of the first portion 806 and the plane 811 of the third portion 810 is between about 2 and 5 inches. In some embodiments, the distance is measured as a vertical distance between one end of the first portion 806 to one end of the third portion 810. In some embodiments, the plane 809 of the second portion 808 is transverse to the plane 807 of the first portion 806 and/or the third portion 810. The second portion 808 may be angled upward from the first portion 806. For example, retractor 800 may include a top surface and a bottom surface, with the bottom surface configured to engage the capitellum. The second portion 808 may extend from the first portion 806 in the direction of the top surface as depicted in FIG. 6A. The third portion 810 may extend from the second portion 808 at bend 814.

In some embodiments, the opening 818 may be in the first portion 806. In some embodiments, the opening 818 may extend between the first portion 806 and the second portion 808 as depicted in FIG. 6B. The opening may have a length of between 0.5 inches and 3 inches.

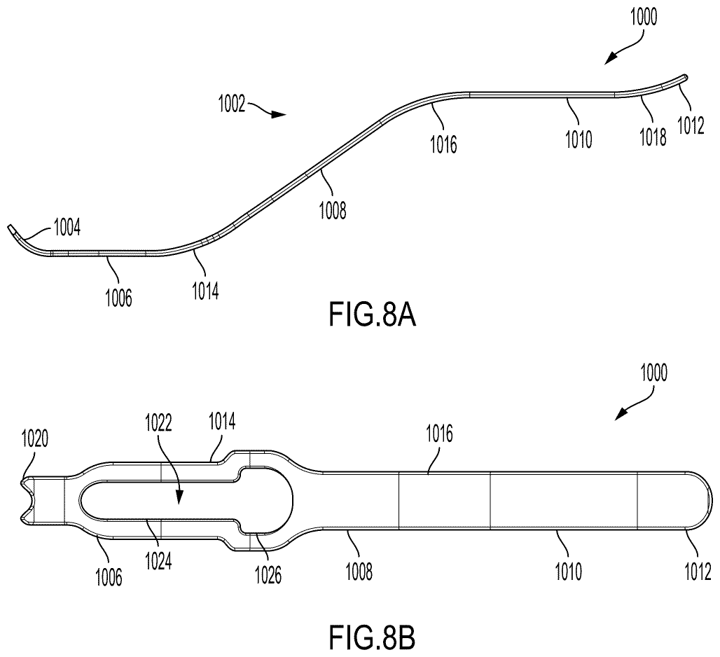

FIG. 7 illustrates retractor 800 in use during a radial head procedure. Opening 818 may be configured to engage the capitellum 604. The end portion 804 may be configured to cradle the radius 602, such as a bottom portion of the radius 602. Applying a force to the elongate body, such as third portion 810 of elongate body, may prop the radius away from the ulna 606 and the capitellum 604. For example, the opening 818 of retractor 800 engages the capitellum and uses the humerus as a leverage point to lift the end portion 504 and the radius 602 away from ulna 606 and out of the incision site. In some embodiments, the force is applied toward the arm to expose the radius 602. As shown in the depicted embodiment, opening 818 of retractor 800 may extend along the elongated body so that reamer 818 or other surgical instrument can be positioned through opening 818 to interact with the radius 602.

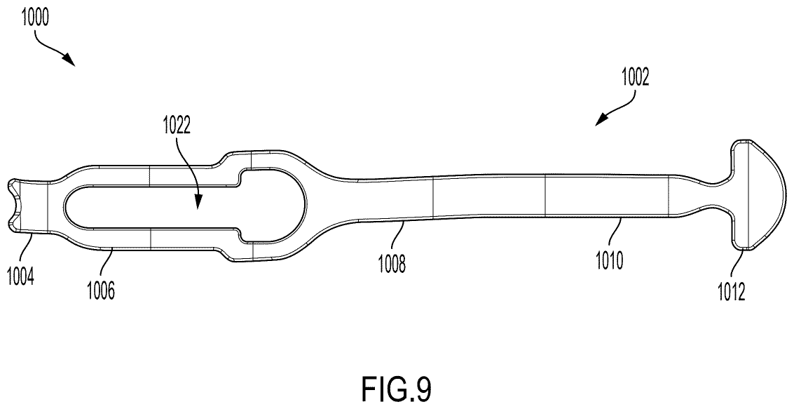

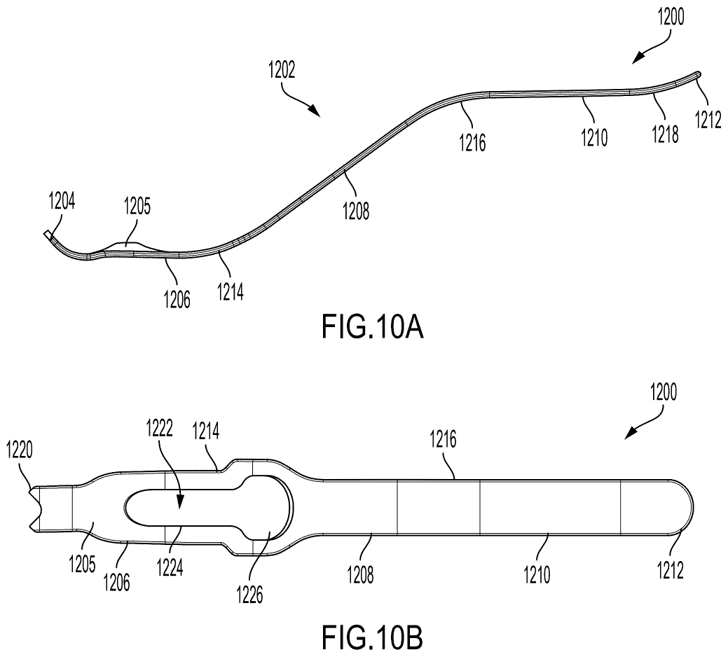

FIGS. 8A, 8B, and 9 illustrate a retractor 1000 according to another example of the present disclosure. The retractor 1000 may include an elongate body 1002, an opening 1022, and an end portion 1004 configured to cradle the radius. The elongate body 1002 and the end portion 1004 of retractor 1000 may be the same as the elongate bodies 102, 302, 504, 802 and end portions 104, 304, 504, 804 of retractors 100, 300, 500, 800 respectively.

In some embodiments, opening 1022 of retractor 1000 may include a first portion 1024 and a second portion 1026 with a greater width than the first portion 1024. The second portion 1026 may be distal to the first portion 1024. The second portion 1026 may be configured to receive a surgical instrument (e.g., a reamer, a bone planar) while the first portion 1024 may be configured to engage the capitellum. In some embodiments, the second portion is located in the second portion 1008 of elongate body (e.g., after bend 1014). While the depicted embodiment shows the first portion 1024 and the second portion 1026 as one opening, in some embodiments, the first portion 1024 and the second portion 1026 are separated into two openings.

The width of the second portion 1026 may be between about 0.15 and 0.45 inches greater than the width of the first portion 1024 of the opening 1022. For example, the second portion 1026 may have a width between 0.65 and 1.05 inches and the first portion 1024 may have a width between 0.2 and 0.9 inches. As shown in the depicted embodiment, the second portion 1026 may be generally in the shape of a semi-circle. However, it can be appreciated that other shapes may be used for the second portion 1026, such as a circle, a triangle, a rectangle, a square, or any suitable shape.

As illustrated in FIGS. 8A and 9, the elongate body includes a fourth portion 1012 in addition to the first portion 1006, the second portion 1008, and the third portion 1010. The fourth portion 1012 may be configured to provide improved leverage for the user. The elongate body 1002 may include a bend 1018 between the third portion 1010 and the fourth portion 1012. The plane extending through the fourth portion 1012 may by transverse to the plane extending through the third portion 1010. Additionally or alternatively, the plane extending through the fourth portion 1012 may be substantially parallel to the plane extending through the second portion 1008. In some embodiments, the fourth portion 1012 may extend upward from the third portion 1010. In other embodiments, the fourth portion 1012 may extend downward from the third portion 1010.

As depicted in FIG. 9, the fourth portion 1012 may include a width greater than the remainder of the elongate body, such as to form a tab on the distal end of retractor 1000. For example, the tab may include a curved edge configured to fit into a hand of a user and a flat edge for grasping with one or more fingers. However, it can be appreciated that other configurations of a tab may be used for the user to grasp. The fourth portion 1012 may provide better leverage for elevating the radius bone.

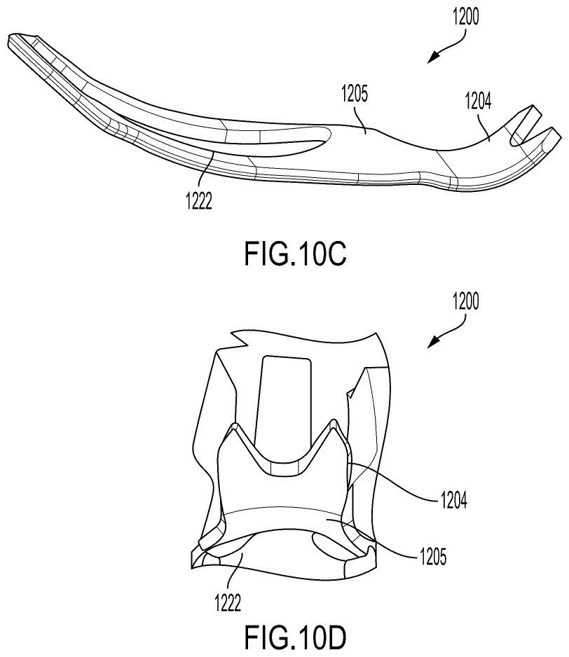

FIGS. 10A through 10D illustrate a retractor 1200 according to another example of the present disclosure. The retractor 1200 may include an elongate body 1202, a feature 1205 for engaging the capitellum, and an end portion 1204 configured to cradle the radius. The elongate body 1202 and the end portion 1204 of retractor 1200 may be the same as the elongate bodies 102, 302, 502, 802, 1002 and end portions 104, 304, 504, 804, 1004 of retractors 100, 300, 500, 800, 1000 respectively.

In some embodiments, retractor 1200 may include an indentation or bump 1205 for engaging the capitellum in addition to or alternative to opening 1222. For example, in the depicted embodiment, a peak of indentation or bump 1205 may be placed proximal to opening 1222, such as between bifurcated end portion 1204 and opening 1222. Indentation or bump 1205 may be configured to receive a portion of the humerus, such as the capitellum. In some embodiments, opening 1214 may be configured to receive a surgical instrument and/or receive a portion of the humerus. However, in some embodiments, the retractor 1200 includes an indentation or bump 1205 without the inclusion of an opening.

The indentation or bump 1205 may be located in the end portion 1204, the elongate body 1202 portion (e.g., the first portion 1206 and/or second portion 1208 of the elongate body 1202), or both the end portion 1204 and the elongate body 1202 of retractor 1200. However, it can be appreciated that the indentation or bump 1205 may be located along other portions of retractor 1200.

As can be seen in FIG. 10A to 10D, the indentation or bump 1205 may extend upward from a top surface of the elongate body 1202. In some embodiments, the indentation or bump 1205 may include a concave surface. As depicted in FIGS. 10C and 10D, in some embodiments, the peak of the concave surface may be proximal to the opening 1222 while a portion of the side may extend into opening 1222. However, in some embodiments, the peak of the concave surface may be within the opening 1222 or the entire concave surface may be fully outside of or within opening 1222. In some embodiments, the concave surface may extend along an entire width of retractor 1202 or may extend along a partial width of the retractor 1202.

The concave surface may be configured to substantially match a surface of the capitellum. When using retractor 1200, a user may position the concave surface of the indentation or bump 1205 over a surface of the capitellum. The concave surface may improve positioning and/or stability of the retractor and result in less stress imparted on the capitellum.

The present disclosure is also directed to methods of retracting a radius using a radial retractor (e.g., 100, 300, 500, 800, 1000, 1200) disclosed herein. In some embodiments, the method includes making an incision on the arm to expose the elbow joint. A user may insert the bifurcated end portion of the retractor under the radius, either before or after a radial head portion has been excised. In some embodiments, inserting the bifurcated end portion under the radius includes positioning the radius between the first protrusion and the second protrusion of the bifurcated end portion to support the radius. In some embodiments, inserting the bifurcated end portion under the radius includes positioning the radius along the bifurcated end portion, such as when using retractor 300.

A user may apply a force on the elongated body of the retractor to elevate the radial bone away from the capitellum and out of the incision site. A user may position the feature of the retractor over the capitellum prior to applying the force to (i) ensure the capitellum is out of the way and (ii) provide extra leverage for elevating the radius bone. The use of the feature may provide stability to the retractor during the procedure.

Once the radius is exposed, a surgical tool (e.g., a reamer or a bone planer) may be used for boring, planning, and/or insertion of a radial head prosthesis. For example, in some embodiments, such as the embodiment illustrated in FIG. 7, the surgical tool may be advanced through an opening in the retractor to reach the radius. Alternatively, the surgical tool may be advanced above the retractor as illustrated in Fig. SB.

### EMBODIMENTS

Various aspects of the subject matter described herein are set out in the following numbered embodiments:
Embodiment 1. A retractor for a radial head procedure, the retractor comprising: an elongated body, the elongated body comprising an opening configured to engage a capitellum; and a bifurcated end portion configured to engage a radius bone, wherein the bifurcated end portion comprises: a first protrusion and a second protrusion, wherein a first free end of the first protrusion and a second free end of the second protrusion are offset from a plane defined by the elongated body.
Embodiment 2. The retractor of embodiment 1, wherein the first protrusion includes a first fixed end connected to the elongated body opposite the first free end, and the second protrusion includes a second fixed end connected to the elongated body opposite the second free end.
Embodiment 3. The retractor of embodiment 2, wherein the first and second protrusions curve from the first fixed end and the second free end to the first free end and the second free end, respectively.
Embodiment 4. The retractor of embodiment 2, wherein the bifurcated end portion defines a plane extending through the first and second fixed ends and the first and second free ends, wherein the plane of the bifurcated end portion is substantially parallel to and offset from the plane of the elongated body.
Embodiment 5. The retractor of any one of embodiments 1-4, wherein a distance between the plane of the elongated body and the first free end and the second free end is between 0.5 inches and 1.0 inches.
Embodiment 6. The retractor of any one of embodiments 1-5, wherein the opening comprises an oval shape.
Embodiment 7. The retractor of embodiment 6, wherein a width of the opening is between 0.4 and 0.6 inches.
Embodiment 8. The retractor of embodiment 6, wherein a length of the opening is between 0.7 and 1.0 inches.
Embodiment 9. The retractor of any one of embodiments 1-8, wherein the opening is positioned at a proximal end portion of the elongated body.
Embodiment 10. The retractor of any one of embodiments 1-9, wherein a width of the elongated body is greatest at the opening.
Embodiment 11. A retractor for a radial head procedure, the retractor comprising: an elongated body, the elongated body comprising: a first portion defining a first plane; a second portion defining a second plane angled relative to the first plane; a third portion defining a third plane angled relative to the second plane; a feature configured to engage a capitellum; and a bifurcated end portion proximate the first portion of the elongated body, the bifurcated end portion configured to engage a radius bone, wherein the bifurcated end portion comprises: a first protrusion and a second protrusion, wherein a first free end of the first protrusion and a second free end of the second protrusion are offset from the first plane.
Embodiment 12. The retractor of embodiment 11, wherein the second plane is angled downward relative to the first plane.
Embodiment 13. The retractor of embodiment 11, wherein the second plane is angled upward relative to the first plane.
Embodiment 14. The retractor of embodiment 11, wherein the feature configured to engage the capitellum comprises an opening.
Embodiment 15. The retractor of embodiment 14, wherein the opening comprises an oval shape.
Embodiment 16. The retractor of embodiment 15, wherein the opening is in the first portion of the elongated body.
Embodiment 17. The retractor of embodiment 14, wherein the opening extends through the first portion and the second portion of the elongated body.
Embodiment 18. The retractor of embodiment 14, wherein the opening comprises an elongated slot.
Embodiment 19. The retractor of embodiment 18, wherein a width of the elongated slot is between 0.4 and 0.6 inches.
Embodiment 20. The retractor of embodiment 14, wherein a first portion of the opening proximate the bifurcated end portion has a first width and a second portion of the opening has a second width greater than the first width.
Embodiment 21. The retractor of any one of embodiments 11-20, wherein the elongated body further comprises a fourth portion comprising a fourth plane angled relative to the third plane.
Embodiment 22. The retractor of embodiment 21, wherein the fourth plane is substantially parallel to the second plane.
Embodiment 23. The retractor of any one of embodiments 11-22, wherein the elongated body comprises a fourth portion distal to the third portion, wherein a largest width of the fourth portion is greater than a largest width of the third portion.
Embodiment 24. The retractor of any one of embodiments 11-23, wherein a second end of the elongated body opposite the bifurcated end portion comprises tabs extending perpendicular to a direction of the elongation of the elongated body.
Embodiment 25. The retractor of any one of embodiments 11-24, wherein the first protrusion includes a first fixed end connected to the elongated body opposite the first free end and the second protrusion includes a second fixed end connected to the elongated body opposite the second free end.
Embodiment 26. The retractor of embodiment 24, wherein the first and second protrusion curve from the first fixed end and the second free end to the first free end and the second free end, respectively.
Embodiment 27. The retractor of any one of embodiments 11-26, wherein the feature configured to engage the capitellum comprises an indentation.
Embodiment 28. The retractor of embodiment 27, wherein the indentation comprises a concave surface.
Embodiment 29. The retractor of embodiment 27, wherein the indentation is in the first portion of the elongated body.
Embodiment 30. The retractor of embodiment 29, wherein the first portion of the elongated body further comprises an opening and the indentation is between the opening and the bifurcated end portion.
Embodiment 31. A method for retracting a radius using a radial retractor, the radial retractor comprising: an elongated body comprising a feature configured to engage a capitellum; and a bifurcated end portion comprising a first protrusion and a second protrusion, wherein a first free end of the first protrusion and a second free end of the second protrusion are offset from a first plane defined by the elongated body, the method comprising: inserting the bifurcated end portion of the radial retractor into an incision site having a radius bone; placing the radial bone between the first protrusion and the second protrusion of the bifurcated end portion of the radial retractor; and applying a force on the radial retractor to elevate the radial bone away from the capitellum, wherein applying the force causes the feature to engage with the capitellum.
Embodiment 32. The method of embodiment 31, wherein the feature engaging with the capitellum provides leverage for elevating the radial bone.
Embodiment 33. The method of any one of embodiments 31 and 32, wherein the feature engaging with the capitellum provides stability of the radial retractor against the capitellum.
Embodiment 34. The method of any one of embodiments 30-33, wherein the radius bone has an excised radial head portion.
Embodiment 35. The method of any one of embodiments 31-34, wherein the elongated body further comprises: a first portion comprising a first plane and an opening; a second portion comprising a second plane angled relative to the first plane; and a third portion comprising a third plane angled relative to the second plane and substantially parallel to the first plane.
Embodiment 36. The method of embodiment 35, wherein the force applied to the radial retractor to elevate the radial bone away from the capitellum is applied to at least the third portion of the elongated body.
Embodiment 37. The method of embodiment 35, wherein the second plane is angled downward relative to the first plane.
Embodiment 38. The method of embodiment 37, further comprising positioning, above the elongated body of the radial retractor, a surgical tool for use on the radial bone while the radial bone is elevated.
Embodiment 39. The method of embodiment 38, wherein the surgical tool comprises one or more of a reamer or a bone planer.
Embodiment 40. The method of embodiment 35, wherein the second plane is angled upward relative to the first plane.
Embodiment 41. The method of embodiment 40, further comprising inserting a surgical tool through an opening on the elongated body for use on the radial bone while the radial bone is elevated.
Embodiment 42. The method of embodiment 41, wherein the surgical tool comprises one or more of a reamer or a bone planer.
Embodiment 43. The method of any one of embodiments 31-42, wherein the feature configured to engage the capitellum comprises a concave surface.

As used herein, “about,” “approximately” and “substantially” are understood to refer to numbers in a range of numerals, for example the range of −10% to +10% of the referenced number, preferably −5% to +5% of the referenced number, more preferably −1% to+1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the system of the present disclosure, there may be terms used like first, second, third, and fourth. These terms may be used for the purpose of differentiating one component from the other, but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A retractor for a radial head procedure, the retractor comprising:
an elongated body, the elongated body comprising an opening configured to engage a capitellum; and
a bifurcated end portion configured to engage a radius bone, wherein the bifurcated end portion comprises:
a first protrusion and a second protrusion, wherein a first free end of the first protrusion and a second free end of the second protrusion are off set from a plane defined by the elongated body.

2. The retractor of claim 1, wherein the first protrusion includes a first fixed end connected to the elongated body opposite the first free end, and the second protrusion includes a second fixed end connected to the elongated body opposite the second free end.

3. The retractor of claim 2, wherein the first and second protrusions curve from the first fixed end and the second free end to the first free end and the second free end, respectively.

4. The retractor of claim 2, wherein the bifurcated end portion defines a plane extending through the first and second fixed ends and the first and second free ends, wherein the plane of the bifurcated end portion is substantially parallel to and offset from the plane of the elongated body.

5. The retractor of claim 1, wherein the opening comprises an oval shape.

6. The retractor of claim 1, wherein the opening is positioned at a proximal end portion of the elongated body.

7. The retractor of claim 1, wherein a width of the elongated body is greatest at the opening.

8. A retractor for a radial head procedure, the retractor comprising:
an elongated body, the elongated body comprising:
a first portion defining a first plane;
a second portion defining a second plane angled relative to the first plane; and
a third portion defining a third plane angled relative to the second plane;
a feature configured to engage a capitellum; and
a bifurcated end portion proximate the first portion of the elongated body, the bifurcated end portion configured to engage a radius bone, wherein the bifurcated end portion comprises:
a first protrusion and a second protrusion, wherein a first free end of the first protrusion and a second free end of the second protrusion are offset from the first plane.

9. The retractor of claim 8, wherein the second plane is angled downward relative to the first plane.

10. The retractor of claim 8, wherein the second plane is angled upward relative to the first plane.

11. The retractor of claim 8, wherein the feature configured to engage the capitellum comprises an opening, the opening comprising an oval shape or an elongated slot.

12. The retractor of claim 11, wherein a first portion of the opening proximate the bifurcated end portion has a first width, and a second portion of the opening has a second width greater than the first width.

13. The retractor of claim 8, wherein the elongated body further comprises a fourth portion comprising a fourth plane angled relative to the third plane.

14. The retractor of claim 13, wherein the fourth plane is substantially parallel to the second plane.

15. The retractor of claim 8, wherein the elongated body comprises a fourth portion distal to the third portion, wherein a largest width of the fourth portion is greater than a largest width of the third portion.

16. The retractor of claim 8, wherein a second end of the elongated body opposite the bifurcated end portion comprises tabs extending perpendicular to a direction of the elongation of the elongated body.

17. The retractor of claim 8, wherein the feature configured to engage the capitellum comprises an indentation.

18. The retractor of claim 17, wherein the indentation is in the first portion of the elongated body.

19. The retractor of claim 18, wherein the first portion of the elongated body further comprises an opening and a peak of the indentation is between the opening and the bifurcated end portion.

20. A method for retracting a radius using a radial retractor, the radial retractor comprising:
an elongated body comprising a feature configured to engage a capitellum; and
a bifurcated end portion comprising a first protrusion and a second protrusion, wherein a first free end of the first protrusion and a second free end of the second protrusion are offset from a first plane defined by the elongated body,
the method comprising:
inserting the bifurcated end portion of the radial retractor into an incision site having a radius bone;
placing the radial bone between the first protrusion and the second protrusion of the bifurcated end portion of the radial retractor; and
applying a force on the radial retractor to elevate the radial bone away from the capitellum, wherein applying the force causes the feature to engage with the capitellum.
